(19) United States
(12) Patent Application Publication
(10) Pub. No.: US 2024/0398930 A1
(43) Pub. Date: Dec. 5, 2024

(54) A VACCINE FOR CORONAVIRUS AND INFLUENZA VIRUS, AND METHOD FOR PREPARATION THEREOF

(71) Applicant: BHARAT BIOTECH INTERNATIONAL LIMITED, Hyderabad (IN)

(72) Inventor: Krishna Murthy ELLA, Hyderabad (IN)

(21) Appl. No.: 18/695,058

(22) PCT Filed: Sep. 24, 2022
(86) PCT No.: PCT/IN2022/050855

§ 371 Date: Mar. 25, 2024

(30) Foreign Application Priority Data
Sep. 24, 2021 (IN) 202141012877

(51) Int. Cl. A61K 39/145; A61K 39/00; A61K 39/215; A61P 37/04; C12N 7/00

(52) U.S. Cl. CPC A61K 39/145; A61K 39/215; A61P 37/04; C12N 7/00; A61K 2039/5254; C12N 2760/16021; C12N 2760/16034; C12N 2760/16052; C12N 2760/16063; C12N 2760/16071; C12N 2770/20034; C12N 2770/20052; C12N 2770/20063; C12N 2770/20071

(57) ABSTRACT

The invention discloses vaccine for coronavirus and influenza virus and method for preparation thereof. More specifically, the invention discloses seasonal viral vaccine i. e. coronavirus and influenza virus vaccine for prophylaxis of novel coronavirus (SARS-CoV-2) infection (COVID-19) and Influenza virus in mammals and method for preparation of such vaccine. The invention discloses the stable combination vaccine compositions of killed-inactivated SARS-CoV-2, Influenza virus (A and B strains) as antigens. The present invention further discloses method of adaptation and growth seasonal influenza (A and B) strains in cell culture and methods of inactivation and purification of influenza virus bulk antigen. The present invention also discloses SARS-CoV-2 vaccine formulation with inactivated Influenza viruses and use of the same to elicit immune response against the SARS-CoV-2 and Influenza viruses in mammals and humans.

### RELATED PATENT APPLICATION(S)

This application claims the priority to and benefit of Indian Provisional Patent Application No. 202141012877 filed on Sep. 24, 2021; the disclosures of which are incorporated herein by reference.

### FIELD OF THE INVENTION

The present invention relates to combination vaccine formulation for coronavirus and quadrivalent influenza (A and B) virus and method for preparation thereof. More specifically, the present invention relates to seasonal viral vaccine i.e. coronavirus and influenza virus vaccine for prophylaxis of novel coronavirus (SARS-CoV-2) infection (COVID-19) and Influenza virus (A and B strains) in mammals and method for preparation of such vaccine. The invention discloses the stable combination vaccine compositions of killed-inactivated SARS-CoV-2, Influenza virus (A and B strains) as antigens. The present invention further discloses method of adaptation and growth of WHO recommended quadrivalent seasonal influenza (A and B) strains in cell culture. The invention also discloses, methods for the preparation of inactivated quadrivalent seasonal influenza (A and B) vaccine and purification of influenza virus bulk antigen. Further the present invention discloses the methods of inactivated SARS-CoV-2 vaccine preparation and formulation with inactivated Influenza viruses (A and B strains) and use of the same to elicit immune response against the SARS-CoV-2 and Influenza viruses (A and B strains) in mammals.

### BACKGROUND OF THE INVENTION

The SARS-CoV-2 is an emerging pathogen, which belongs to the genus Betacoronavirus of family Coronaviridae. The genome is linear single-stranded positive sense RNA of approximately 29 Kilobases in size. The genome analysis reveals that the SARS-CoV-2 is closely related to the bat coronavirus than the SARS-CoV or MERS-COV. The SARS-CoV-2 emerged from Wuhan, China and had spread rapidly throughout the world. The disease caused by the SARS-CoV-2 is named as COVID-19. As per the World Health Organization, till 20 Nov. 2020, more than 56,623,643 confirmed human infections were reported with more than 1,355,963 deaths. These numbers are greater than that of SARS and MERS combined. In the COVID-19 outbreak, it was observed that the transmission of the SARS-CoV-2 happen before the appearance of the symptoms. This attributed to the increased number of human infections. The lack of protective immunity against coronaviruses makes the SARS-CoV-2 a potential threat.

Influenza viruses are classified under the genus Orthmyxoviridae. There are four types of influenza viruses, namely, A, B, C and D. Among these, the Influenza A and B strains cause outbreaks and spreads around the world in yearly outbreaks, with about three to five million human cases of severe illness and about 3-6 hundred thousand deaths. The influenza virus spreads through the air from coughs or sneezes from infected person.

Since, the SARS-CoV-2 and Influenza viruses transmit through similar mode, it is predicted that the COVID-19 become seasonal and an increase in the COVID-19 cases can be expected during winter.

Currently, there are no licensed human vaccine available for the combination of COVID-19 and seasonal influenza. Licensed vaccines are available for seasonal influenza. The present patent application describes the preparation and formulation of a combination vaccine to prevent SARS-CoV-2 and Influenza virus (A and B strains) infections.

Bharat Biotech International Limited (BBIL) procured four seasonal Influenza strains as recommended by the World Health Organization (WHO) for the influenza season 2022-23; from University of Wisconsin-Madison, USA i.e, A/Wisconsin/588/2019 (H1N1); A/Darwin/6/2021 (H3N2); B/Austria/1359417/2021; B/Phuket/3073/2013. In the context of the disclosure herein, the vaccine methods and formulation developed using any one of the Influenzas virus strains is applicable all Influenza virus strains for use as candidate vaccine.

### OBJECTIVES OF THE INVENTION

The followings are the objectives of the present invention:

The main objective of the invention is to provide vaccine formulations for prophylaxis against SARS-CoV-2 and quadrivalent seasonal influenza (A and B).

Another objective of the invention is to provide method of adaptation and growth of WHO recommended quadrivalent seasonal influenza (A and B) strains in MDCK cells.

Another object of the invention is to provide methods for the preparation of inactivated quadrivalent seasonal influenza (A and B) vaccine and purification of Influenza virus bulk antigen.

One more object of the invention is to provide methods for quadrivalent seasonal influenza (A and B) inactivation by chemical means i.e, formalin and beta propiolactone in presence or absence of stabilizers.

Another objective of the invention is to provide method of manufacture of a combination vaccine with or without adjuvants to prevent SARS-CoV-2 and Influenza virus (A and B) infections.

Further objective of the invention is to provide combination vaccine formulation comprising; SARS-CoV-2 virus at dose concentration of at least 6 ug/dose with Inactivated Influenza virus (A & B) virus antigens, at dose concentration of at least 15 ug/dose with or without an adjuvant, either as a single dose or in two or more doses to elicit an immune response for combination vaccine.

Further objective is to check the safety, efficacy and potency of combination vaccine of killed inactivated SARS-CoV-2, and Inactivated Influenza virus (A and B) strains.

Still, a further objective is to provide a method to administer the antigen through intranasal, oral, intramuscular, subcutaneous, and intradermal routes.

### SUMMARY OF THE INVENTION

The present invention relates to vaccine for coronavirus and influenza virus and method for preparation thereof. More specifically, the present invention relates to seasonal viral vaccine i. e. coronavirus and influenza virus vaccine formulation for prophylaxis of novel coronavirus (SARS-CoV-2) infection (COVID-19) and Influenza virus (A and B) strains in mammals and method for preparation of such vaccine.

The invention discloses a combined vaccine formulation comprising whole virion inactivated SARS-Cov-2 and influenza (A and B) antigens formulated with adjuvants in pharmaceutically acceptable buffer, wherein vaccine formulation elicits protective response against each of viruses in mammals.

In one aspect of the invention, wherein quadrivalent influenza (A and B) antigen is prepared using MDCK cells as cell substrate by adapting the virus to MDCK cells.

In one embodiment of the invention, quadrivalent influenza (A and B) antigen is purified and concentrated antigen obtained by clarification of the viral harvest using membrane filtration, followed by purification by column chromatography; and tangential flow filtration using membranes with cut off from 100 kDa.

In the above-mentioned embodiment, column chromatography elutes majority of the virus antigen in the flow through such as Capto Core 700, most preferably Capto Core 700 wherein the virus sample is purified on Capto Core 700 column and is eluted in the flow through.

In another embodiment of the invention, quadrivalent seasonal influenza (A and B) is inactivated by chemical means i.e, formalin or beta propiolactone or combination of both in presence or absence of stabilizers

In the above-mentioned embodiment, the inactivation of quadrivalent influenza [A and B] Influenza virus is carried out before or after purification of the virus.

In another aspect of the invention, Influenza A virus or influenza B virus were inactivated by one of the following methods selected from:
a) Formalin treatment at any concentration ranging from 1:500 up to 1:4000 v/v of formalin: virus, at 8° C. to 37° C., preferably 25±3° C., for at least 1 to 7 days;
b) Formalin treatment at any concentration ranging from 1:500 up to 1:4000 v/v of formalin: virus, at 2° C. to 8° C. for at least 10 to 30 days;
c) Beta-propiolactone at any concentration ranging from 1:500 up to 1:4000 v/v of BPL: virus, for at least 24 to 48 hrs at temperatures ranging from 8° C. to 30° C., preferably 25±3° C., for 48 hrs;
d) Beta-propiolactone at any concentration ranging from 1:500 up to 1:4000 v/v of BPL: virus, at 2° C. to 8° C. for at least 3-7 days;
e) A combination of BPL and formalin at any aforementioned conditions, preferably BPL inactivation at 1:3000 (PBL: virus v/v) for 24 hrs followed by formalin inactivation at 1:3000 (formalin: virus, v/v) for 24 to 48 hrs at 15° C. to 30° C., preferably 25±3° C.

In another aspect of the invention wherein the inactivation of Influenza (A and B) virus is carried out in the absence or presence of a stabilizing agent, wherein the stabilizing agent is 1% sorbitol and 0.5% L-glycine.

Another one aspect of the invention, the vaccine formulation comprises of one or more ingredient/components such as adjuvants, pH adjusting agents, buffers, preservatives, and other pharmaceutically acceptable excipients suitable for vaccine or any combination thereof.

The said combined vaccine formulation contains SARS-CoV-2 whole virion antigen (BBV152) at dose concentration of at least 6 ug/dose in 0.5 mL volume.

The said combined vaccine formulation contains whole virion Influenza (A and B) antigens at dose concentration of at least 15 ug/dose in 0.5 mL volume.

The said combined vaccine formulation further contains adjuvant Algel-IMDG comprising of 250 ug-350 ug of Al³⁺ concentration per dose and 15 ug-30 ug of TLR7/8 agonists per dose in 0.5 mL volume.

The said combined vaccine formulation is formulated in Phosphate buffer is phosphate at concentration of 5 mM up to 200 mM of Phosphate ions of any PH between 7 to PH 8.

The said combined vaccine formulation further comprises 2-phenoxyethanol as preservative at the concentration of 1 to 5 mg/ml.

Another aspect of the invention involves, method of preparing a combined vaccine formulation comprising whole virion inactivated SARS-Cov-2 and influenza [A and B] antigens formulated with or without adjuvants in pharmaceutically acceptable buffer; Wherein dose concentration of SARS-CoV-2 whole virion antigen (BBV152) is at least 6 ug/dose in 0.5 mL volume and dose concentration of each whole virion Influenza [A and B] antigens is at least 15 ug/dose in 0.5 mL volume.

In the said method, the adjuvant used is Algel-IMDG, containing 250 ug-350 ug of Al³⁺ concentration and 15 ug-30 ug of TLR 7/8 agonist per dose in 0.5 ml volume.

In the said method, vaccine is formulated in phosphate buffer at the concentration of 5 mM up to 200 mM at PH between 7 to PH 8.

The said method, formulation further involves the 2-phenoxyethanol as the preservative at concentration of 1 mg/ml to 5 mg/ml.

Yet another aspect of the invention, involves use of combined vaccine formulation to induce robust immune response against SARS-Cov-2 and influenza [A and B] infection.

Yet in another aspect of the invention, the combined vaccine formulation can be administered through intranasal, oral, intramuscular, subcutaneous, and intradermal routes.

### BRIEF DESCRIPTION OF FIGURES

### Immune Response:

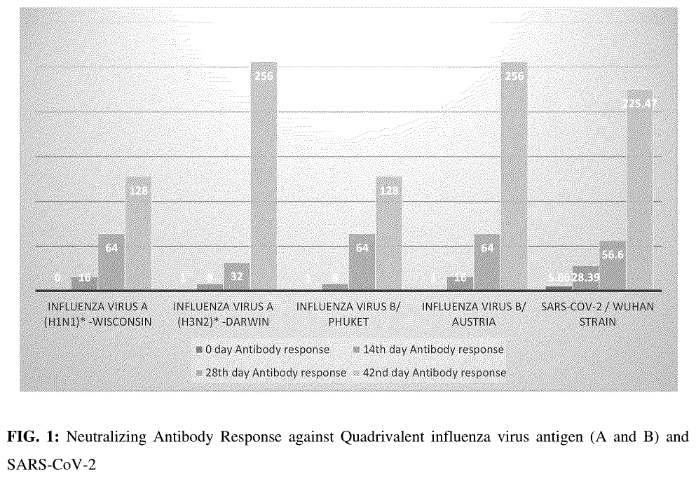

FIG. 1: Neutralizing Antibody Response against Quadrivalent influenza virus antigen (A and B) and SARS-CoV-2.

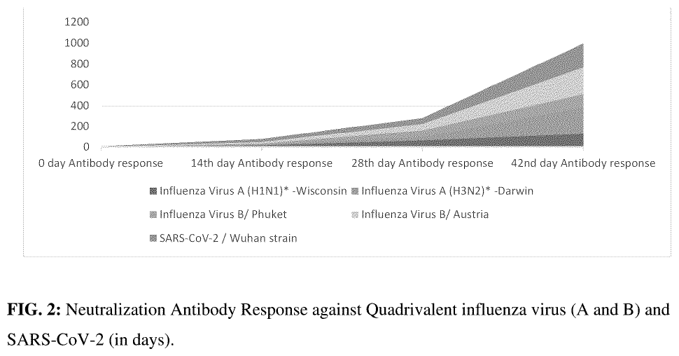

FIG. 2: Neutralization Antibody Response against Quadrivalent influenza virus (A and B) and SARS-CoV-2 (in days).

### DETAILED DESCRIPTION OF THE INVENTION

The present invention relates to vaccine for coronavirus and influenza virus and method for preparation thereof. More specifically, the present invention relates to seasonal viral vaccine i. e. coronavirus and influenza virus vaccine composition and/or formulation for prophylaxis of novel coronavirus (SARS-CoV-2) (COVID-19) and Quadrivalent Influenza (A and B) infection in mammals and method for preparation of such vaccine.

The invention discloses the stable vaccine compositions of killed-inactivated SARS-CoV-2 and killed inactivated Quadrivalent Influenza virus (A and B) as antigens. The present invention also relates to the methods of inactivated Influenza viruses (A and B) and SARS-CoV-2 vaccine preparation and use of the same to elicit immune response against the SARS-CoV-2 and Influenza viruses (A and B strains) in mammals and humans.

### Vaccine Composition:

The killed-inactivated purified SARS-CoV-2 bulk and Quadrivalent Influenzas [A and B] bulk is used as an active ingredient in the preparation of an immunogenic composition or vaccine composition and the said composition can be used to prevent disease and/or infection with SARS-CoV-2 and Influenza.

In one embodiment the said immunogenic composition comprises antigens as an active ingredient along with pharmaceutically acceptable excipients.

The thus produced vaccine candidate is further provided as an immunogenic composition or vaccine composition comprising an immunogenically effective concentration of vaccine candidate sufficient to elicit desires immunogenic result with or without one or more pharmaceutically acceptable excipients(s).

The vaccine composition of the invention is obtained by a process wherein antibodies are largely elicited against the SARS-CoV-2 and Quadrivalent Influenza (A and B) such as in optimally inactivated virus

### Antigen:

The present invention discloses the stable vaccine composition of killed-inactivated SARS-CoV-2 virus and Quadrivalent Influenza [A and B] as antigen.

### SARS-CoV-2:

The novel corona virus (SARS-CoV-2) belongs to the genus Betacoronavirus of family Coronaviridae. The genome is linear single-stranded positive sense RNA of approximately 29 Kilobases in size. Coronaviruses are enveloped, spherical in shape and are about 120 nm in diameter. As the genome is positive sense, it is infectious and acts as viral messenger RNA. Coronavirus viruses are found in a wide variety of animals and can cause respiratory and enteric disorders of diverse severity. The genome analysis reveals that the SARS-CoV-2 is closely related to the bat coronavirus than the SARS-CoV or MERS-COV.

Coronaviruses are associated with common cold in humans with typically mild to moderate symptoms also called as common cold. These infections usually subside without causing serious health problems. However, the outbreaks of Severe Acute Respiratory Syndrome (SARS) and Middle East Respiratory Syndrome (MERS) have together resulted in more than 10,000 human infections with more than 1,500 fatalities. In distinct contrast to the mild human coronaviruses, infection with SARS-CoV frequently resulted in severe symptoms including fever, dry cough, shortness of breath and pneumonia.

### Influenza A and B:

The Influenzas virions are enveloped and usually spherical. It is composed of a viral envelope, matrix proteins and viral ribonucleocapsids (vRNPs). There are four types of influenza viruses: A, B, C and D. Human influenza A and B viruses cause seasonal epidemics of disease (known as flu season). Influenza pandemic can occur when a new and different influenza virus emerges that both infects people and has the ability to spread efficiently among people. Influenza C virus infections generally cause mild illness and are not known to cause human epidemics. Influenza D viruses primarily affect cattle and are not known to infect or cause illness in people.

Influenza A virus belongs to the genus Alphainfluenzavirus of the virus family Orthomyxoviridae. The genome of influenza A and Influenza B virus consists of eight single-stranded, negative-sense RNAs that are associated with multiple copies of nucleoprotein and three viral RNA polymerase subunits to form the viral ribonucleoprotein complexes (vRNPs).

Influenza A viruses are divided into subtypes based on two proteins on the surface of the virus: hemagglutinin (H) and neuraminidase (N). There are 18 different hemagglutinin subtypes and 11 different neuraminidase subtypes (H1 through H18 and N1 through N11, respectively). While more than 130 influenza A subtype combinations have been identified in nature, primarily from wild birds, there are potentially many more influenza A subtype combinations given the propensity for virus “reassortment. Current subtypes of influenza A viruses that routinely circulate in people include: A(H1N1) and A(H3N2).

Influenza A(H1N1) viruses are related to the pandemic 2009 H1N1 virus that emerged in the spring of 2009 and caused a flu pandemic. These viruses have continued to circulate seasonally since then and have undergone genetic changes and changes to their antigenic properties that affect immunity. Influenza A(H3N2) viruses also change both genetically and antigenically.

Influenza B virus belongs to genus Betainfluenza virus of the family Orthomyxoviridae. Influenza B viruses are not divided into subtypes, but instead are further classified into two lineages: B/Yamagata and B/Victoria. Influenza B viruses generally change more slowly in terms of their genetic and antigenic properties than influenza A viruses, especially influenza A(H3N2) viruses.

Since, SARS-COV-2 and Influenza viruses transmit through similar mode, present patent application describes the preparation and formulation of a combination vaccine to prevent SARS-CoV-2 and Influenza virus (A and B strains) infections.

A cell line that can be propagated in vitro culture can be used as a host for Influenza (A and B) virus culture. For propagating Influenza (A and B) strains, preferably permissive cells which allow the virus to grow well are selected. Madin-Darby Canine Kidney (MDCK) cells was used as cell substrate for the culture of the Influenza (A and B) virus. MDCK cells were grown in in DMEM (Dulbecco's Modified Eagle Medium; containing 10% of New Born Fetal Serum (NBFS) and incubated at 35° C. to 37° C. until reaching 80-100% confluence of the monolayer. Post-infection, the same medium was used, or alternatively the virus was cultured in MDCK cells was adapted to serum free medium. Influenza virus was cultured routinely in MDCK cells. Influenza virus strains A/Wisconsin/588/2019 (H1N1), A/Darwin/6/2021 (H3N2), B/Austria/1359417/2021 and B/Phuket/3073/2013 was adapted to MDCK cells by direct inoculation in MDCK cells and transferred the cell stacks to walk In incubator for virus adsorption at 36±1° C. for 1 Hr±15 min. Influenza virus produces cytopathic effect (CPE) in MDCK cells, and at the optimal Multiplicity of Infection (MOI) and harvest conditions, virus titers above 10e8.5 TCID50/mL were be attained.

For Influenza virus culture at pilot scale, the virus culture was systematically scaled up from T-175 flasks to CS 1 (cell stack 1), CS 10 (cell stack 10) and CS40 (cell stack 40). Multiples of CS40 simultaneously infected with the virus at standardized Mol was used to scale up production. The harvest volume from 5×CS40's was approximately 40 L.

For maintenance in cell culture of the above-mentioned cell lines, MDCK cells in particular, stationary culture in monolayers, perfusion system culture, shake flasks, roller tube/bottle culture, suspension culture with and without microcarriers, cell factories and cell stacks, bioreactors and disposable bioreactors, wave bioreactors and the like can be adopted. For example, various types of microcarriers are commercially available. Commercially available animal cell culture devices can be used to facilitate the growth of cells to high cell density.

The viral harvest was clarified with 0.80+0.45 μM Capsule filter. The clarified harvest volume was around 37.5 L. The clarified viral harvest was then passed through Capto Core700 column (GE healthcare Life Sciences) in phosphate buffered saline (10 mM PB with 50 mM NaCl), pH 7.3. Clarified harvest was loaded on to the column at a flow rate of 300 mL/min and collected the flow through in a 50 LPP bottle the maximum OD at 280 was 1.424. The Virus was further concentrated with Cassette of 100 kDa. The concentrated virus fraction was used for virus inactivation. Influenza sample was inactivated (killed) by various methods for use as vaccine antigens.

Formalin inactivation was tested at various concentrations ranging from 1:1000 (formalin: virus, v/v) to 1:4000 (formalin: virus, v/v) at temperature 25+5 C, more specifically at 22° C. and the kinetics of virus inactivation was monitored every 24 hours for up to 10 days, and routinely the virus inactivation was carried out at 25+3° C., preferably at 22 C for 7 days. The virus inactivation was effective at all concentrations from 1:1000 v/v formalin: virus, up to 1:3500 v/v formalin: virus, at the aforementioned temperatures and time intervals. A ratio of 1:4000 v/v of formalin: virus was effective in virus inactivation at higher temperatures up to 30 to 37° C. for 3 to 7 days. Formalin inactivation was effective at all the aforementioned ratios of formalin to virus at temperatures ranging from 2-8° C. when incubated for time intervals longer than 10 days. Hence formalin inactivation offers flexibility of virus inactivation at any temperature from 2° C. to 37° C. at time intervals ranging from 24 hours to more than 10 days depending upon the conditions used for inactivation. Influenza virus inactivation with Beta propiolactone (BPL) was tested under various conditions. Influenza virus was completely inactivated at BPL concentrations ranging from 1:1000 (BPL: virus, v/v) up to 1:3500 (BPL: virus, v/v) at temperatures from 25+5° C. for 24 to 48 hours. At higher concentration of BPL or at higher temperatures up to 37° C. complete inactivation was achieved in 24 hours or less, and can be used as a method for quick inactivation of the virus. Influenza virus could also be inactivated at the aforementioned concentrations of BPL when incubated at 2 to 8° C. for 3 to 7 days. A combination of BPL inactivation at 1:3500 (BPL: virus, v/v) at 22-25° C. for 48 hours, followed by treatment with low concentrations of formalin from 1:3000 to 1:4000 v/v of formalin: virus for 24 hours was effective in both inactivating and stabilizing the virus. Any concentration of BPL and formalin could be used for both inactivation and stabilizing the virus, as long as inactivation is complete without deleterious effect on immunogenicity. All the above inactivation methods were carried out in the presence and absence of virus stabilizing agents by adding the stabilizer 55 mL of Glycine (0.5%) with 44 mL of Sorbitol (1%) to the 2.2 L of Influenza Strain concentrated and filtered retentate.

### Antigenic Concentration:

The dose concentration of SARS-CoV-2 whole virion antigen (BBV152) is at least 6 ug/dose, whereas dose concentration of each whole virion Quadrivalent Influenza [A and B] antigens is at least 15 ug/dose in 0.5 mL volume.

### Excipient(s):

The composition comprising killed-inactivated SARS-CoV-2 and Quadrivalent Influenzas A and B of the present invention generally may comprise and/or formulated with or without one or more pharmaceutically acceptable excipient(s), suitable for vaccine composition or formulation to be administered in mammals through various routes of administration in suitable concentration.

The vaccine composition of the invention may further comprise an adjuvant, wherein the adjuvant is selected from the group consisting of a) aluminum salts comprising aluminum hydroxide, aluminum phosphate, aluminum sulphate phosphate; b) inulin; c) algammulin which is a combination of inulin and aluminium hydroxide; d) monophosphoryl lipid A (MPL); e) resiquimod; f) muramyl dipeptide (MDP); g) N-glycolyl dipeptide (GMDP); h) 50 polyIC; i) CpG oligonucleotide; j) aluminum hydroxide with MPL; k) any water in oil emulsion; 1) any oil in water emulsion that contains one or more of the following constituents: squalene or its analogues or any pharmaceutically acceptable oil, tween-80, sorbitan trioleate, alpha-tocopherol, cholecalciferol and aqueous buffer, or any of the analogues and derivatives of the molecules thereof j) Algel-IMDG i) two or more combination of any of the aforementioned adjuvants when formulated with SARS-CoV-2 virus and Influenzas antigens enhance the immune response against the virus.

In one preferred embodiment, composition comprises, Algel-IMDG containing 250 ug-750 ug of Al³⁺ concentration, 15 ug-25 ug of TLR7/8 agonists per dose in 0.5 ml Volume.

The adjuvant of the composition of the invention confers immunity when administrated in mammals.

Further the composition may contain the excipients and preservatives.

The vaccine composition of the invention optionally comprises 2-phenoxyethanol as preservative at a concentration of 1 to 5 mg/mL.

The said immunogenic composition comprises Antigen: active ingredient inactivated, purified SARS-CoV-2 and Influenza virus in buffer comprising of Phosphate buffered saline.

In another embodiment of the invention, the candidate vaccine can be administered either as a single dose or in two or more doses by intranasal, intraperitoneal, oral, intramuscular, subcutaneous or intradermal routes in animals and humans to elicit the immune response.

In another embodiment of the invention, assays for neutralizing antibody titers were conducted to check the neutralizing antibody levels against combined vaccine formulations of the present invention which has shown to elicit the high level of neutralizing antibodies.

The combination vaccine comprising SARS-CoV-2 antigen at the dose concentration of at least 6 ug/dose and each strain of Quadrivalent Influenza [A and B] antigens is administrated at the dose concentration of at least 15 ug/dose in 0.5 ml volume; with or without adjuvants either as a single dose or in two or more doses to elicits an immune response.

### Administration

The combined vaccine formulation comprising inactivated SARS-CoV-2 and inactivated Quadrivalent Influenza (A and B) vaccine formulation can be administered to animals and humans through intranasal, intraperitoneal, oral, intramuscular, subcutaneous or intradermal routes.

In one embodiment of the invention, the present vaccine formulation can be administered either as a single dose or in two or more doses by intranasal, intraperitoneal, oral, intramuscular, subcutaneous or intradermal routes in animals and humans to elicit the immune response.

In another embodiment of the invention, assays for antibody titres were conducted to check the antibody levels against vaccine formulations of the present invention which has shown to elicit the high-level end titers response.

### Method of Treatment

In another aspect the invention discloses a method of treatment and/or prophylaxis for COVID-19 and Quadrivalent Influenza [A and B], wherein the said method comprises administration of immunogenic composition by various routes.

In one embodiment the invention provides a method of eliciting a protective immune response in mammals comprising administering the vaccine composition of by any route comprising intramuscular, intradermal, subcutaneous, intravenous, oral, intranasal or transcutaneous routes.

The composition of the invention may be administered by any method comprising needles and syringes including pre-filled syringes, microneedle patch, needle-free patch, inhalation and nasal sprays.

The vaccine composition, immunogenic composition and method of treatment as described above, wherein vaccine composition contains the dose concentration of SARS-CoV-2 whole virion antigen (BBV152) of at least 6 ug/dose in 0.5 mL volume and dose concentration of each whole virion Influenza [A and B] antigens of at least 15 ug/dose in 0.5 mL volume.

In one of the preferred embodiments, present invention provides a method of treatment and/or prophylaxis for COVID-19 and Influenza [A and B] and/or eliciting an immune response against SARS-CoV-2 and Influenza [A and B] in mammals including human subjects, wherein the said method comprises intramuscular administration of a vaccine formulation, wherein vaccine composition contains the dose concentration of SARS-CoV-2 whole virion antigen (BBV152) around at least 6 ug/dose in 0.5 mL volume and dose concentration of each whole virion Influenza [A and B] antigens around at least 15 ug/dose in 0.5 mL volume.

### Advantages

To provide the combined immunogenic vaccine formulation against pandemic potential SARS-CoV-2 and Quadrivalent Influenza (A and B) virus strains.
To provide the combined immunogenic vaccine formulation against mutation prone SARS-CoV-2 and Quadrivalent Influenza (A and B) WHO recommended strains.
To provide the combined immunogenic vaccine formulation against SARS-CoV-2 and Quadrivalent Influenza (A and B), viruses having similar mode of transmission.
To provide the immunogenic vaccine formulation against wherein antigenic response against SARS-CoV-2 and Quadrivalent Influenza (A and B) is elicited without any antigenic interference and cross neutralization.

### EXAMPLES

### Example 1: Formulation

Combination of Inactivated Quadrivalent Influenza (A and B) and SARS-CoV-2 virus antigen. The 2022-2023, WHO recommended influenzas A and B strains were procured from University of Wisconsin-Madison, USA.

| | Seasonal Influenza vaccine strains |
|---|---|
| Influenza A | A/Wisconsin/588/2019 (H1N1) |
| | A/Darwin/6/2021 (H3N2) |
| Influenza B | B/Austria/1359417/2021 |
| | B/Phuket/3073/2013 |

The Influenza A virus strains mentioned in the above table contains six segments (1, 2, 3, 5, 7 and 8) of A/Puerto Rico/8HY/1934 and two segments of WHO recommended H1N1 or H3N2 (segment 4 for Hemagglutinin and segment 6 for Neuraminidase), respectively. The Influenza B virus strains mentioned in the above table contains six segments (1, 2, 3, 5, 7 and 8) of B/Yamagata/1/73 and two segments of WHO recommended B/Austria/1359417/2021 & B/Phuket/3073/2013 (segment 4 for Hemagglutinin and segment 6 for Neuraminidase), respectively. In the context of the disclosure herein, the vaccine methods and formulation developed using any one of the Influenzas virus strains is applicable all Influenza virus strains for use as candidate vaccine.

### Example 2: Influenza [A and B] Virus Culture in MDCK Cells

Madin-Darby Canine Kidney (MDCK) cells was used as cell substrate for the culture of the Influenza (A and B) virus. MDCK cells were grown in in DMEM (Dulbecco's Modified Eagle Medium; containing 10% of New Born Fetal Serum (NBFS) and incubated at 35° C. to 37° C. until reaching 80-100% confluence of the monolayer. Post-infection, the same medium was used, or alternatively the virus was cultured in MDCK cells was adapted to serum free medium. Influenza virus was cultured routinely in MDCK cells. Influenza virus strains A/Wisconsin/588/2019 (H1N1), A/Darwin/6/2021 (H3N2), B/Austria/1359417/2021 and B/Phuket/3073/2013 was adapted to MDCK cells by direct inoculation in MDCK cells and transferred the cell stacks to walk In incubator for virus adsorption at 36±1° C. for 1 Hr±15 min. Influenza virus produces cytopathic effect (CPE) in MDCK cells, and at the optimal Multiplicity of Infection (MOI) and harvest conditions, virus titers above 10e8.5 TCID50/mL were be attained.

### Example 3: Influenza Virus Purification

For Influenza virus culture at pilot scale, the virus culture was systematically scaled up from T-175 flasks to CS 1 (cell stack 1), CS 10 (cell stack 10) and CS40 (cell stack 40). Multiples of CS40 simultaneously infected with the virus at standardized Mol was used to scale up production. The harvest volume from 5×CS40's was approximately 40 L.

The viral harvest was clarified with 0.80+0.45 μM Capsule filter. The clarified harvest volume was around 37.5 L. The clarified viral harvest was then passed through Capto Core700 column (GE healthcare Life Sciences) in phosphate buffered saline (10 mM PB with 50 mM NaCl), pH 7.3. Clarified harvest was loaded on to the column at a flow rate of 300 mL/min and collected the flow through in a 50 LPP bottle the maximum OD at 280 was 1.424. The Virus was further concentrated with Cassette of 100 kDa. The concentrated virus fraction was used for virus inactivation.

### Example 4: Influenza Virus Inactivation

Influenza sample was inactivated (killed) by various methods for use as vaccine antigens.

Formalin inactivation was tested at various concentrations ranging from 1:1000 (formalin: virus, v/v) to 1:4000 (formalin: virus, v/v) at temperature 25+5 C. more specifically at 22° C. and the kinetics of virus inactivation was monitored every 24 hours for up to 10 days, and routinely the virus inactivation was carried out at 25+3° C., preferably at 22 C for 7 days. The virus inactivation was effective at all concentrations from 1:1000 v/v formalin: virus, up to 1:3500 v/v formalin: virus, at the aforementioned temperatures and time intervals. A ratio of 1:4000 v/v of formalin: virus was effective in virus inactivation at higher temperatures up to 30 to 37° C. for 3 to 7 days. Formalin inactivation was effective at all the aforementioned ratios of formalin to virus at temperatures ranging from 2-8° C. when incubated for time intervals longer than 10 days. Hence formalin inactivation offers flexibility of virus inactivation at any temperature from 2° C. to 37° C. at time intervals ranging from 24 hours to more than 10 days depending upon the conditions used for inactivation. Influenza virus inactivation with Beta propiolactone (BPL) was tested under various conditions. Influenza virus was completely inactivated at BPL concentrations ranging from 1:1000 (BPL: virus, v/v) up to 1:3500 (BPL: virus, v/v) at temperatures from 25+5° C. for 24 to 48 hours. At higher concentration of BPL or at higher temperatures up to 37° C., complete inactivation was achieved in 24 hours or less, and can be used as a method for quick inactivation of the virus. Influenza virus could also be inactivated at the aforementioned concentrations of BPL when incubated at 2 to 8° C. for 3 to 7 days. A combination of BPL inactivation at 1:3500 (BPL: virus, v/v) at 22-25° C. for 48 hours, followed by treatment with low concentrations of formalin from 1:3000 to 1:4000 v/v of formalin: virus for 24 hours was effective in both inactivating and stabilizing the virus. Any concentration of BPL and formalin could be used for both inactivation and stabilizing the virus, as long as inactivation is complete without deleterious effect on immunogenicity.

All the above inactivation methods were carried out in the presence and absence of virus stabilizing agents by adding the stabilizer 55 mL of Glycine (0.5%) with 44 mL of Sorbitol (1%) to the 2.2 L of Influenza Strain concentrated and filtered retentate.

### Example 5: Preparation of Combination Formulation

The combined vaccine formulation was prepared by mixing at least 15 ug/dose of each strain of Inactivated Quadrivalent Influenza [A and B] virus and at least 6 ug/dose of Inactivated SARS-CoV-2 virus in 0.5 mL volume. All antigens were formulated with Algel IMDG comprising 250 ug-350 μg of Al³⁺ and 15 ug-30 μg of TLR7/8 agonists (Imidazo Quinoline Gallamide (IMDG), 2-phenoxyethonol (2.5 mg) in phosphate buffer saline up to 0.5 ml.

### Example 5.1: Physiochemical Characterization of Formulation

Formulation was characterized for quantification of HA and total protein expression, particle size by Lowry method, ELISA, western blot and Zeta Sizer and formulation was found to be stable.

### Example 5.2: Animals

6-8-week-old, Balb/c mice (both male and female) were purchased and maintained in the animal care facility under standard approved protocols. All procedures involving mice were carried out with the approval of Institutional Animal Ethics Committee.

### Example 6: Immunization

### Example 6.1: Enzyme Linked Immunosorbent Assay (ELISA)

Two set of 6-8 weeks old Balb/C mice (Both Male and Female) were used for immunization to determine the antibody titer by ELISA. Each set contained Test and Control group. The test group received 1/10^{th} of human intended dose, whereas control group were only administrated PBS (Phosphate buffer saline). The doses were given two times to one set of mice at the interval of 0 and 14th day through intramuscular (IM route) whereas another set was administrated immunogenic formulation through intraperitoneal route (IP route) two times at the interval of 0 and 7^{th} day. The mice was partially bleed by retro orbital plexus at 14 days after first and second dose (IM Group) and 7 days after first and second dose (IP group). The sera were collected at different time points were pooled group wise and used to determine end time titres.

The results indicated that combined vaccine formulation elicited high titer range for Anti SARS-CoV 2 IgG antibodies against spike S1 protein; for the IM test group in range 6400 to 12800 (IM group) and 400 to 3200 (IP group) (as shown in table 1).

TABLE 1
Spike (S1) specific End point Antibody Binding Titer

| | IM (Group) | IP (Group) |
|---|---|---|
| Post 7/14 days after | 6400-12800 | 400-3200 |
| first and second dosage | | |

### Example 6.2: Hemagglutination Inhibition (HI) Assay and Micro-Neutralization Antibody Titers (MNT50)

In this example, mice were vaccinated to evaluate the immunogenicity of a combination vaccine formulations (at least 15 μg Influenza [A and B], 6 μg of SARS-CoV-2 antigen concentration). For this, mice were administered intramuscularly with full HSD (full human intended single dose) of combination vaccine formulations containing at least 15 μg/SHD and 6 μg/SHD antigen concentration/0.5 ml/mice) on day 0 and 28. Blood was collected on various time points, either before the immunization (Day 0) or 14 Days post immunization (Day 14, 28 and 42). Sera was separated and stored at −20° C. until further use for immunogenicity analysis.

### Example 6.2.1

### Hemagglutination Inhibition (HI) Assay and the HI Titre:

In this experiment, Hemagglutination-inhibition (HI) assay was performed to identify the hemagglutinin (HA) subtype of an unknown isolate or the HA subtype specificity of antibodies to Inactivated Influenza virus. The sera sample collected were diluted 2-fold in PBS. After sera dilution 4HAU/25 ul or 8HAU/50 ul was added in each well, following incubation Human RBC's (0.25%) was added to observe the highest sera dilution showing button formation. The antibody titer is expressed as the reciprocal of the highest serum dilution showing complete inhibition using 4 HAU units/25 μL or 8 HAU units/50 L.

The results indicated that after the second dose of the combined vaccine formulation, the immune response and sera-conversion above minimum threshold for sero-protection was observed for all four Quadrivalent Influenza (A and B) strain. (represented in Table 2; FIGS. 1 and 2)

### Example 6.2.2

### Micro-Neutralization Antibody Titers (MNT50):

After the dosing schedule on 0 day, 28th day in Balb/c by the IM, the individual sera collected from all groups on Day 0, 14, 28 and 42 was used to test neutralization antibody titers by MNT50. The sera were diluted from 1:8 unto 1:1024 using MEM (minimum essential medium), by 2 fold sera dilution method. The 100 CCID₅₀ was added for virus neutralization; further the Vero cell suspension (1*10⁵ cells/ml) was added and incubated for 5 to 7 days to study the cytopathic effect and highest dilution showing 50% well protection. The reading was calculated using Reed and Muench method.

These results indicated that after the 2^{nd} dose of combined vaccine formulation the immune response and robust sera conversion was observed above threshold level against SARS-CoV-2 antigen (represented in Table 2; FIGS. 1 and 2).

TABLE 2

| | | | Correlates of |
|---|---|---|---|
| | Hemagglutination- | | protection Minimum |
| | inhibition | Microneutralization | Threshold for |
| Sample Details | (HI) assay | Test at 50% | Seroprotecting |
| COVIFLU | 0 day: HI titre 0 | Not Applicable | HI titre ≥40 |
| VACCINE (20 | 14^{th} day: HI titre 16 | | |
| mL Batch Size) | 28^{th} day: HI titre 64 | | |
| Flu strain 1 | 42^{nd} day: HI titre 128 | | |
| COVIFLU | 0 day: HI titre 1 | Not Applicable | HI titre ≥40 |
| VACCINE (20 | 14^{th} day: HI titre 8 | | |
| mL Batch Size) | 28^{th} day: HI titre 32 | | |
| Flu strain 2 | 42^{nd} day: HI titre 256 | | |
| COVIFLU | 0 day: HI titre 1 | Not Applicable | HI titre ≥40 |
| VACCINE (20 | 14^{th} day: HI titre 8 | | |
| mL Batch Size) | 28^{th} day: HI titre 64 | | |
| Flu strain 3 | 42^{nd} day: HI titre 128 | | |
| COVIFLU | 0 day: HI titre 1 | Not Applicable | HI titre ≥40 |
| VACCINE (20 | 14^{th} day: HI titre 16 | | |
| mL Batch Size) | 28^{th} day: HI titre 64 | | |
| Flu strain 4 | 42^{nd} day: HI titre 256 | | |
| COVIFLU | Not Applicable | 0 day: 5.66 | ≥22.50 |
| VACCINE (20 | | 14^{th} day: 28.39 | |
| mL Batch Size) | | 28^{th} day: 56.60 | |
| SARS CoV2 | | 42^{nd} day: 225.47 | |
| (NIV-2020-770 | | | |
| strain) | | | |

### Example 6.3: Safety Evaluation

BALB/c mice administered with two doses of vaccine Formulation found safe with no mortality during the entire experimental period. No abnormal clinical signs, no abnormal body weight gain noticed. Feed consumption was normal.

In conclusion based on Immunogenicity data it is confirmed that combination vaccine of SARS CoV2 and four recommended influenza vaccine strain by WHO is providing sero-protection by matching with correlates of protection and seroconversion occurring post vaccination with dose schedule of 0 and 28 day and antigen concentration is proving good immune response.

We claim:

1. A combined vaccine formulation comprising whole virion inactivated SARS-Cov-2 and quadrivalent influenza [A and B] antigens formulated with or without adjuvants in pharmaceutically acceptable buffer, wherein vaccine formulation elicits protective response against each of viruses in mammals.

2. The vaccine formulation as claimed in claim 1, wherein quadrivalent influenza (A and B) antigen is prepared using MDCK cells as cell substrate by adapting the virus to MDCK cells.

3. The vaccine formulation as claimed in claim 1, wherein said quadrivalent influenza (A and B) antigen is a purified and concentrated antigen obtained by clarification of the viral harvest using membrane filtration, followed by purification by column chromatography; and tangential flow filtration using membranes with cut off from 100 kDa.

4. The vaccine formulation as claimed in claim 3, wherein said purification by column chromatography comprises gel filtration, mixed mode resin column chromatography, ion exchange column chromatography, affinity matrix chromatography and hydrophobic interaction chromatography.

5. The vaccine formulation as claimed in claim 4, wherein the column chromatography elutes majority of the virus antigen in the flow through such as Capto Core 700, most preferably Capto Core 700 wherein the virus sample is purified on Capto Core 700 column and is eluted in the flow through.

6. The vaccine formulation as claimed in claim 1, wherein the quadrivalent influenza [A and B] antigens are inactivated by at least one or more of a chemical inactivating agent.

7. The vaccine formulation as claimed in claim 6, wherein the inactivation of Influenza virus is carried out before or after purification of the virus.

8. The vaccine formulation as claimed in claim 7, wherein the quadrivalent influenza [A and B] antigen is inactivated by chemical inactivating agent selected from formalin (formaldehyde), beta propiolactone (BPL).

9. The vaccine formulation as claimed in claim 8, Influenza A virus or influenza B virus were inactivated by one of the following methods selected from:
a) Formalin treatment at any concentration ranging from 1:500 up to 1:4000 v/v of formalin: virus, at 8° C. to 37° C., preferably 25±3° C., for at least 1 to 7 days;
b) Formalin treatment at any concentration ranging from 1:500 up to 1:4000 v/v of formalin: virus, at 2° C. to 8° C. for at least 10 to 30 days;
c) Beta-propiolactone at any concentration ranging from 1:500 up to 1:4000 v/v of BPL: virus, for at least 24 to 48 hrs at temperatures ranging from 8° C. to 30° C., preferably 25±3° C., for 48 hrs;
d) Beta-propiolactone at any concentration ranging from 1:500 up to 1:4000 v/v of BPL: virus, at 2° C. to 8° C. for at least 3-7 days;
e) A combination of BPL and formalin at any aforementioned conditions, preferably BPL inactivation at 1:3000 (PBL: virus v/v) for 24 hrs followed by formalin inactivation at 1:3000 (formalin: virus, v/v) for 24 to 48 hrs at 15° C. to 30° C., preferably 25±3° C.

10. The vaccine formulation as claimed in claim 1, wherein the inactivation of Influenza (A and B) virus is carried out in the absence or presence of a stabilizing agent.

11. The vaccine formulation as claimed in claim 10, wherein the stabilizing agent is 1% sorbitol and 0.5% L-glycine.

12. The vaccine formulation as claimed in claim 1, wherein dose concentration of SARS-CoV-2 whole virion antigen (BBV152) is at least 6 ug/dose in 0.5 mL volume.

13. The vaccine formulation as claimed in claim 1, wherein dose concentration of whole virion Influenza [A and B] antigens is at least 15 ug/dose in 0.5 mL volume.

14. The vaccine formulation as claimed in claim 1, wherein adjuvant is Algel-IMDG.

15. The vaccine formulation as claimed in claim 14, wherein Algel IMDG comprises 250 ug-350 ug of Al³⁺ concentration per dose in 0.5 ml.

16. The vaccine formulation as claimed in claim 14, wherein Algel IMDG comprises 15 ug-30 ug of TLR7/8 agonists per dose in 0.5 ml.

17. The vaccine formulation as claimed in claim 1, wherein pharmaceutically acceptable buffer is phosphate at concentration of 5 mM up to 200 mM of Phosphate ions of any PH between 7 to PH 8.

18. The vaccine formulation as claimed in claim 1, wherein formulation further comprise preservative.

19. The vaccine formulation as claimed in claim 18, wherein preservative is 2-phenoxyethanol.

20. The vaccine formulation as claimed in claim 19, wherein the concentration of 2-phenoxy ethanol in the formulation is 1 to 5 mg/ml.

21. A method of preparing a combined vaccine formulation comprising whole virion inactivated SARS-Cov-2 and influenza [A and B] antigens formulated with or without adjuvants in pharmaceutically acceptable buffer; Wherein dose concentration of SARS-CoV-2 whole virion antigen (BBV152) is at least 6 ug/dose in 0.5 mL volume; Wherein dose concentration of whole virion Influenza [A and B] antigens is at least 15 ug/dose in 0.5 mL volume.

22. The method as claimed in claim 21, wherein quadrivalent influenza (A and B) antigen is prepared using MDCK cells as cell substrate by adapting the virus to MDCK cells.

23. The method as claimed in claim 21, wherein said quadrivalent influenza (A and B) antigen is a purified and concentrated antigen obtained by clarification of the viral harvest using membrane filtration, followed by purification by column chromatography; and tangential flow filtration using membranes with cut off from 100 kDa.

24. The method as claimed in claim 23, wherein said purification by column chromatography comprises gel filtration, mixed mode resin column chromatography, ion exchange column chromatography, affinity matrix chromatography and hydrophobic interaction chromatography.

25. The method as claimed in claim 24, wherein the column chromatography elutes majority of the virus antigen in the flow through such as Capto Core 700, most preferably Capto Core 700 wherein the virus sample is purified on Capto Core 700 column and is eluted in the flow through.

26. The method as claimed in claim 21, wherein the quadrivalent influenza [A and B] antigens are inactivated by at least one or more of a chemical inactivating agent.

27. The method as claimed in claim 26, wherein the inactivation of Influenza virus is carried out before or after purification of the virus.

28. The method as claimed in claim 26, wherein the quadrivalent influenza [A and B] antigen is inactivated by chemical inactivating agent selected from formalin (formaldehyde), beta propiolactone (BPL).

29. The vaccine formulation as claimed in claim 28, Influenza A virus or influenza B virus were inactivated by one of the following methods selected from:
a) Formalin treatment at any concentration ranging from 1:500 up to 1:4000 v/v of formalin: virus, at 8° C. to 37° C., preferably 25±3° C., for at least 1 to 7 days;
b) Formalin treatment at any concentration ranging from 1:500 up to 1:4000 v/v of formalin: virus, at 2° C. to 8° C. for at least 10 to 30 days;
c) Beta-propiolactone at any concentration ranging from 1:500 up to 1:4000 v/v of BPL: virus, for at least 24 to 48 hrs at temperatures ranging from 8° C. to 30° C., preferably 25±3° C., for 48 hrs;
d) Beta-propiolactone at any concentration ranging from 1:500 up to 1:4000 v/v of BPL: virus, at 2° C. to 8° C. for at least 3-7 days;
e) A combination of BPL and formalin at any aforementioned conditions, preferably BPL inactivation at 1:3000 (PBL: virus v/v) for 24 hrs followed by formalin inactivation at 1:3000 (formalin: virus, v/v) for 24 to 48 hrs at 15° C. to 30° C., preferably 25±3° C.

30. The method as claimed in claim 21, wherein the inactivation of Influenza (A and B) virus is carried out in the absence or presence of a stabilizing agent.

31. The method as claimed in claim 30, wherein the stabilizing agent is 1% sorbitol and 0.5% L-glycine.

32. The method as claimed in claim 21, wherein adjuvant is Algel-IMDG.

33. The method as claimed in claim 32, wherein Algel IMDG comprises 250 ug-350 ug of Al³⁺ concentration per dose in 0.5 ml.

34. The method as claimed in claim 32, wherein Algel IMDG comprises 25 ug-30 ug of TLR7/8 agonists per dose in 0.5 ml.

35. The method as claimed in claim 21, wherein pharmaceutically acceptable buffer is phosphate at concentration of 5 mM up to 200 mM of Phosphate ions of any PH between 7 to PH 8.

36. The method as claimed in claim 21, wherein formulation further comprise preservative.

37. The method as claimed in claim 36, wherein preservative is 2-phenoxyethanol.

38. The method as claimed in claim 37, wherein the concentration of 2-phenoxy ethanol in the formulation is 1 to 5 mg/ml.

39. The method as claimed in claim 21, wherein combined vaccine formulation is administered through intranasal, oral, intramuscular, subcutaneous, and intradermal routes.

40. The use of vaccine formulation as claimed in claim 1, to induce robust immune response against SARS-Cov-2 and influenza [A and B] infection.
